(19) United States
(12) Patent Application Publication
(10) Pub. No.: US 2017/0189655 A1
(43) Pub. Date: Jul. 6, 2017

(54) SYSTEM AND METHOD FOR DRAINING CEREBROSPINAL FLUID

(71) Applicant: Health Research, Inc., Buffalo, NY (US)

(72) Inventor: Andrew Fabiano, Buffalo, NY (US)

(21) Appl. No.: 15/313,129

(22) PCT Filed: May 27, 2015
(86) PCT No.: PCT/US15/32764

§ 371 Date: Nov. 22, 2016

Related U.S. Application Data
(60) Provisional application No. 62/003,068, filed on May 27, 2014.

(51) Int. Cl. A61M 27/00; A61M 39/10

(52) U.S. Cl. CPC A61M 27/006; A61M 39/1011; A61M 2210/1085

(57) ABSTRACT

A system and/or a method of evacuating cerebrospinal fluid (CSF) from a CSF reservoir is provided. The system can include a CSF reservoir, a reservoir mating piece, and a securing piece. The reservoir mating piece can have a tip end configured to be insertable into the CSF reservoir and a drainage end configured to be coupled to a drainage tube, the reservoir mating piece forming a drainage passageway for the evacuation of CSF from the CSF reservoir. The securing piece can be configured to be affixed to an individual's skin. In an unlocked position, the securing piece may be configured to slide along the reservoir mating piece, and in a locked position, the securing piece may be configured to be fixed relative to the reservoir mating piece. At least a portion of the drainage passageway is nonlinear between the tip end and the drainage end.

### CROSS-REFERENCE TO RELATED APPLICATIONS

This application claims priority to U.S. Provisional Application No. 62/003,068, filed on May 27, 2014, now pending, the disclosure of which is incorporated herein by reference.

### FIELD OF THE DISCLOSURE

The present disclosure relates generally to draining cerebrospinal fluid from a body, such as an individual.

### BACKGROUND OF THE DISCLOSURE

Individuals with neurosurgical conditions often have a cerebrospinal fluid (CSF) reservoir implanted for drug delivery or as part of a CSF diversion system such as a shunt. Conditions such as hydrocephalus, can cause high intracranial pressures which must often be relieved before continued treatment. This is sometimes accomplished by the shunt built in to the reservoir. However, failure can occur within components of a CSF shunt, for example due to a mechanical disruption/obstruction, and generally requires the replacement of the failed component(s). Valves in CSF reservoirs can fail due to debris build-up (e.g., blood, protein) within the valve, and the outlet of the catheter can fail by fracturing, becoming obstructed, or tethering within scar tissue. These mechanical failures, infections, and other complications cause many implanted CSF shunts to fail within a relatively short period of time.

Where a shunt has failed or a reservoir does not have a shunt, relief of intracranial pressure, is sometimes performed by externally draining a CSF reservoir by repeatedly tapping the reservoir using a syringe and withdrawing CSF, as shown in FIG. 1. Alternatively, an external ventricular drainage system can be placed for the collection of CSF in order to mitigate the high intracranial pressure. However, external ventricular drains require placement of a ventricular catheter and the correspondent risks with such an invasive procedure. CSF can also be drained by directly withdrawing CSF from the ventricles.

There is a need for alternative methods for relieving intracranial pressure through less invasive techniques.

### SUMMARY OF THE DISCLOSURE

In one embodiment, the disclosure is embodied as a system for evacuating cerebrospinal fluid (CSF) by way of a CSF reservoir. The system can include a CSF reservoir, a reservoir mating piece, and a securing piece. The reservoir mating piece can have a tip end configured to be insertable into the CSF reservoir and a drainage end configured to be coupled to a drainage tube, the reservoir mating piece forming a drainage passageway for the evacuation of CSF from the CSF reservoir. The securing piece can be configured to be affixed to an individual's skin. In an unlocked position, the securing piece may be configured to slide along the reservoir mating piece, and in a locked position, the securing piece may be configured to be fixed relative to the reservoir mating piece. At least a portion of the drainage passageway is nonlinear between the tip end and the drainage end.

In another embodiment, the disclosure is embodied as a method of evacuating CSF from a CSF reservoir. The method can include implanting a CSF reservoir into an individual, or the CSF reservoir may have already been implanted in a previous procedure. A tip end of a reservoir mating piece can be inserted into the CSF reservoir. The reservoir mating piece can form a drainage passageway for the evacuation of CSF from the CSF reservoir. A drainage end of the CSF reservoir can be coupled to an evacuation assembly. A securing piece can be moved from a first position relative to the reservoir mating piece to a second position relative to the reservoir mating piece. The securing piece can be locked in the second position such that the securing piece is fixed relative to the reservoir mating piece. The securing piece may be secured to the individual's skin. CSF from the CSF reservoir can be drained through the drainage passageway of the reservoir mating piece and through the evacuation assembly. At least a portion of the drainage passageway is nonlinear between the tip end and the drainage end.

### DESCRIPTION OF THE DRAWINGS

For a fuller understanding of the nature and objects of the disclosure, reference should be made to the following detailed description taken in conjunction with the accompanying drawings, in which:

FIG. 1 depicts a prior art system; and

FIG. 2 depicts a system according to an embodiment of the disclosure;

FIG. 3 depicts a device according to an embodiment of the disclosure; and

FIG. 4 depicts a method according to an embodiment of the disclosure.

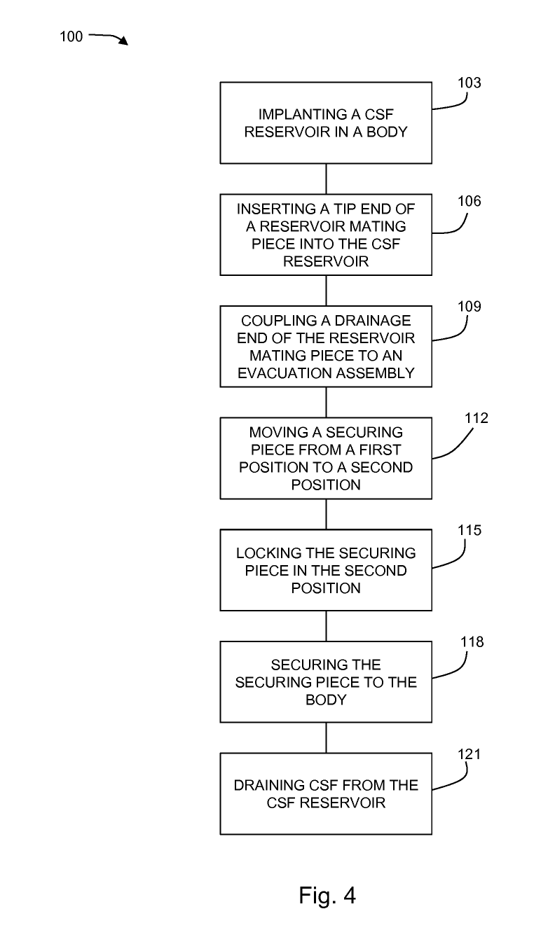

### DETAILED DESCRIPTION OF THE DISCLOSURE

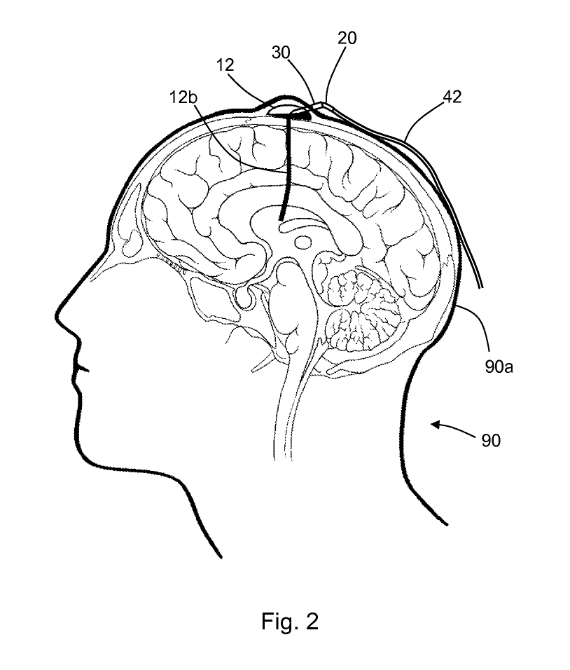

FIG. 2 depicts a system 10 for evacuating CSF from a CSF reservoir 12 implanted in an individual 90. The system 10 can include a reservoir mating piece 20, a securing piece 30, and an evacuation assembly 40.

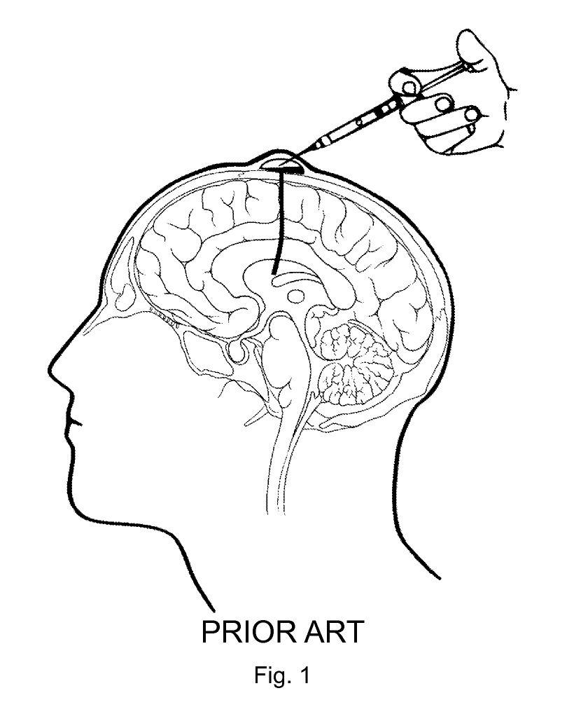

A CSF reservoir 12 such as a conventional ventriculostomy reservoir, for example an Ommaya reservoir depicted in FIG. 1, may reside in place under the scalp of an individual. The CSF reservoir 12 can be used for the aspiration of CSF or for the delivery of drugs (e.g., chemotherapeutic agents) into the CSF. The CSF reservoir 12 can include a catheter 12b in a lateral ventricle attached to the reservoir 12b implanted under the scalp of the individual 90. The CSF reservoir 12 may include a base having a catheter connector, an integral, upwardly extending cylindrical wall portion, and a flange portion integrally formed with and overlying the wall portion. The base may be manufactured from a metal or other suitable material. A cap, which can be made of a silicone elastomer material or other suitable materials, is typically provided to enclose the upper end of the base and define, with the base, an internal reservoir. A surgeon disposes the catheter into the ventricle through the skull, and attaches the catheter to the connector at the lower end of the base, positioning the reservoir under the scalp. Once the incision is closed, the reservoir can subsequently be accessed through the skin to obtain CSF or deliver medications directly to the CSF.

In a system 10 of the present disclosure, the reservoir mating piece 20 forms a drainage passageway for the evacuation of CSF from the CSF reservoir 12. The reservoir mating piece 20 can have a tip end 22 configured to be insertable into the CSF reservoir 12 and a drainage end 24 configured to be coupled to a drainage tube 42 of an evacuation assembly 40. In this manner, CSF can flow from the CSF reservoir 12, through the reservoir mating piece 20 and drainage tube 42, and into an external vessel 44, such as a bag.

The tip end 22 of the reservoir mating piece 20 may have, for example, a needle-shape for puncturing an individual's skin 90a, and/or other tissue, to access an implanted CSF reservoir 12. The reservoir mating piece 20 can have a curved portion 26 to more closely follow an outer contour of an individual (e.g., the head of the individual). In this way, the reservoir mating piece 20 can be secured closer to the head of the individual for longer-term use (e.g., up to 1-7 days or longer). The reservoir mating piece 20 may be manufactured in multiple sizes and/or curvatures such that a suitable configuration can be selected for a particular individual. Disposition of the reservoir mating portion 20 proximate to the head via the curved portion 26 may also serve to prevent kinking once an appropriate dressing is applied to the entry point. Thus, at least a portion of the drainage passageway in the reservoir mating piece 20 can be nonlinear. At least a portion of the reservoir mating piece 20 may be made of a rigid material, such as a metal. In one embodiment, the entire reservoir mating piece 20 is made of an 18 gauge or 25 gauge stainless steel. The length between the tip end 22 and the curved portion 26 may be approximately 3 cm, but different lengths can be selected according to factors such as patient age, and a patient's scalp thickness. The length between the curved portion 26 and the drainage end 24 can be about 2 cm, but, here again, other lengths may be used according to the application.

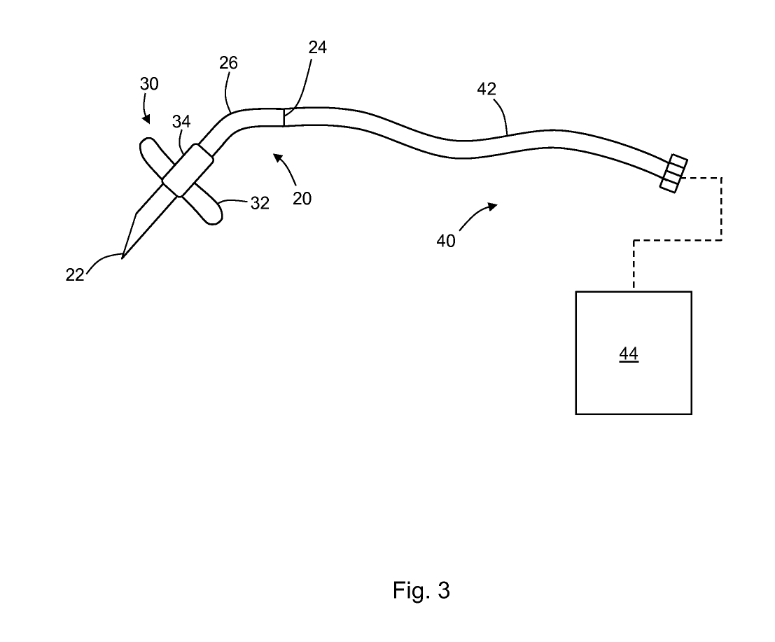

The securing piece 30 can include a mating portion 32 and an adjustment mechanism 34. The mating portion 32 can be configured to affix to an individual's skin 90a. In one embodiment, the mating portion 32 includes an adhesive for securing the securing piece 30 to an individual's skin 90a. In another embodiment, the mating portion 32 may be suture compatible, for example, having plastic flanges for securing the securing piece 30 to an individual's skin 90a with sutures (see, for example, FIG. 3). The mating portion 32 is preferably a pliable material that can form to the contour of the individual's 12 skull. Other forms of attachment to the skin will be apparent in light of the present disclosure and are included within the scope of the present disclosure. In one example, the securing piece 30 can be disc-shaped with a diameter of 5 cm.

The position of the securing piece 30 can be movable relative to the reservoir mating piece 20 via the adjustment mechanism 34. In an “unlocked” configuration, the position of the securing piece 30 can be adjusted relative to the reservoir mating piece 20. In a “locked” configuration, the securing piece 30 is configured to be fixed relative to the reservoir mating piece 20. Thus, the adjustment mechanism 34 can be a slidably lockable mechanism that slides along the reservoir mating piece 20 for adjustment purposes, and then locks into position for use with an individual. In an embodiment, the adjustment mechanism 34 is a sleeve which can be locked into position by, for example, a suture tied around a circumference of the sleeve. Those skilled in the art will appreciate that various adjustment mechanisms may be used with the reservoir mating piece 20, including mechanisms having screw-connections or clamping connections.

In a locked position, the securing piece 30 can be located between the tip end 22 and the curved portion 26 of the reservoir mating piece 20. As such, the reservoir mating piece 20 may be inserted, through the individual's scalp, into the reservoir 12, and the securing piece 30 moved to a position proximate to the skin of the individual 90a. The securing piece 30 can then be secured to the skin 90a of the individual and locked to a position on the reservoir mating piece 20 to hold the system 10 in place. The adjustability of the system 10 allows for a particular individual's need. For example, the position of the securing piece 30, relative to the reservoir mating piece 20, can be adjusted to accommodate a particular type or size of CSF reservoir 12, location of CSF reservoir 12 in an individual 90, and size of an individual 90. A more reliable and comfortable placement of the reservoir mating piece 30 can be thereby be achieved.

In use, a CSF reservoir 12 can be implanted into an individual 90 according to known methods (or has already been implanted during a previous procedure). The tip end 22 of the reservoir mating piece 20 can be inserted into the CSF reservoir 12. The securing piece 30 can be moved from a first position relative to the reservoir mating piece 20 to a second position relative to the reservoir mating piece 20. The securing piece 30 may be secured to the individual's skin. The securing piece 30 can be locked in the second position such that the securing piece 30 is fixed relative to the reservoir mating piece 20. A drainage end 24 of the reservoir mating piece 20 can be coupled to an evacuation assembly 40. The drainage end 24 may have been connected to the drainage assembly 40 before insertion of the reservoir mating piece 20. An external vessel 44 (which may or may not form a portion of the system 10) may be attached to the drainage assembly by, for example, a luer connector. CSF from the CSF reservoir 12 can be drained through the drainage passageway of the reservoir mating piece 20, through the evacuation assembly 40, into the external vessel 44. The drainage end 24 may additionally, or alternatively, be connected to an intracranial pressure monitoring device. At least a portion of the drainage passageway may be nonlinear between the tip end and the drainage end.

Once the desired procedure is performed, the tip end 22 of the reservoir mating piece 20 can be removed from the CSF reservoir 12. The CSF reservoir 12 may be self-sealing, such that once the tip end 22 is removed, the CSF reservoir 12 remains operable.

In another aspect of the present disclosure, a method 100 for evacuating CSF is provided. The method 100 includes implanting 103 a CSF reservoir into a body. The step of implanting 103 a CSF reservoir may include inserting a catheter into a lateral ventricle of the body. A tip end of a reservoir mating piece is inserted 106 into the CSF reservoir. For example, the tip end may be used to pierce the skin of the body and a wall of the CSF reservoir. In this way, the reservoir mating piece is disposed through the skin and the wall of the CSF reservoir and forms a drainage passageway for the evacuation of CSF from the CSF reservoir.

A drainage end of the reservoir mating piece is coupled 109 to an evacuation assembly. For example, the drainage end may be configured as a locking luer taper, configured to couple 109 with a corresponding connector of a drain tube of an evacuation assembly. A securing piece of the reservoir mating piece is moved 112 from a first position relative to the reservoir mating piece to a second position. In an embodiment, the securing piece is configured to slide along a length of the reservoir mating piece from the first position (at a location along the length of the reservoir mating piece) to the second position (at another location along the length of the reservoir mating piece).

The securing piece is locked 115 in the second position. In this way, the securing piece is fixed relative to the reservoir mating piece. The securing piece is secured 118 to the body. In this way, the reservoir mating piece can be fixed to the skin by way of the securing piece—thereby reducing the risk that the reservoir mating piece is unintentionally removed from the CSF reservoir. It should be noted that locking the securing piece may be a reversible procedure such that the securing piece can be unlocked for removal or adjustment of the reservoir mating piece.

The method 100 includes draining 121 CSF from the CSF reservoir through the drainage passageway formed by the reservoir mating piece and into the evacuation assembly.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. A system for evacuating cerebrospinal fluid (CSF), comprising:
a CSF reservoir configured to be implanted below the skin of an individual;
a reservoir mating piece having a tip end configured to be insertable into the CSF reservoir and a drainage end configured to be coupled to a drainage tube, the reservoir mating piece forming a drainage passageway for the evacuation of CSF from the CSF reservoir;
a securing piece configured to be affixed to the skin of the individual, wherein in an unlocked position, the securing piece is configured to slide along the reservoir mating piece, and in a locked position, the securing piece is configured to be fixed relative to the reservoir mating piece; and
wherein at least a portion of the drainage passageway is nonlinear between the tip end and the drainage end.

2. The system of claim 1, wherein the securing piece is a disk disposed circumferentially around the reservoir mating piece, the disk having an adhesive side for attachment to the skin.

3. The system of claim 1, wherein the securing piece comprises at least one flange.

4. The system of claim 1, wherein the securing piece is configured to be locked in the locked position by suture.

5. The system of claim 1, wherein the drainage end of the reservoir mating piece comprises a luer lock configured to correspond to an end of the drainage tube.

6. A device for evacuating CSF from a CSF reservoir, comprising:
a reservoir mating piece having a tip end configured to be insertable into the CSF reservoir and a drainage end configured to be coupled to a drainage tube, the reservoir mating piece forming a drainage passageway for the evacuation of CSF from the CSF reservoir;
a securing piece configured to be affixed to an individual's skin, wherein in an unlocked position, the securing piece is configured to slide along the reservoir mating piece, and in a locked position, the securing piece is configured to be fixed relative to the reservoir mating piece; and
wherein at least a portion of the drainage passageway is nonlinear between the tip end and the drainage end.

7. The system of claim 6, wherein the securing piece is a disk disposed circumferentially around the reservoir mating piece, the disk having an adhesive side for attachment to the skin.

8. The system of claim 6, wherein the securing piece comprises at least one flange.

9. The system of claim 6, wherein the securing piece is configured to be locked in the locked position by suture.

10. The system of claim 6, wherein the drainage end of the reservoir mating piece comprises a luer lock configured to correspond to an end of the drainage tube.

11. The system of claim 6, further comprising an evacuation assembly.

12. The system of claim 11, wherein the evacuation assembly comprises a drainage tube and a bladder in fluid communication with the drainage tube, wherein the drainage tube is configured to couple with the reservoir mating piece, thereby extending the drainage passageway from the CSF reservoir to the bladder.

13. A method for evacuating CSF, comprising:
implanting a CSF reservoir into a body;
inserting a tip end of a reservoir mating piece into the CSF reservoir, the reservoir mating piece forming a drainage passageway for the evacuation of CSF from the CSF reservoir;
coupling a drainage end of the reservoir mating piece to an evacuation assembly;
moving a securing piece from a first position relative to the reservoir mating piece to a second position relative to the reservoir mating piece;
locking the securing piece in the second position such that the securing piece is fixed relative to the reservoir mating piece;
securing the securing piece to the body; and
draining CSF from the CSF reservoir through the drainage passageway of the reservoir mating piece and into the evacuation assembly; and
wherein at least a portion of the drainage passageway is nonlinear between the tip end and the drainage end.
